(19) United States
(12) Patent Application Publication
(10) Pub. No.: US 2006/0093515 A1
(43) Pub. Date: May 4, 2006

(54) Automated solution generator

(76) Inventors: Adam M. Fermier, Easton, PA (US); Kelly Swinney, Bethlehem, PA (US); Benjamin Young, Sheffield, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/980,468

(22) Filed: Nov. 3, 2004

(51) Int. Cl. C12Q 1/68

(52) U.S. Cl. 422/50

(57) ABSTRACT

The present invention relates to an automated solution generator, such as for use in generating buffering or clinical formulations or mobile phase solutions for chromatography systems, and methods for operating the automated solution generator to accurately dispense solutions, perform dilutions and prepare solutions with reduced user intervention.

### FIELD OF THE INVENTION

The present invention relates to an automated solution generator, such as for use in generating buffering or clinical formulations or mobile phase solutions for chromatography systems, and methods for operating the automated solution generator to accurately dispense solutions, perform dilutions and prepare solutions with reduced user intervention.

### BACKGROUND

Separation science is a universal technique that benefits most scientists. Chromatographic separations provide a means to determine to what extent a reaction has yielded the desired products, to monitor impurities and dissolution profiles, and to study degradation pathways in drug products. Heretofore, conventional analytical HPLC separation devices consist of a flow-metering pump to control volumetric flow rates of solvent gradients. Either low or high pressure mixing is used to produce the gradient. As the names imply, low pressure and high pressure mixing refer to the forming of the gradient either pre- or post-pump, respectively. An essential part of any chromatographic separation process is the preparation of precise mobile phases or solutions.

It is not unusual for a research laboratory to require many liters of mobile phases of varying composition on a daily basis. Such preparations are typically done manually to ensure accuracy of solution compositions. In order to increase lab productivity, a system for preparing HPLC mobile phase/mixed solutions or dispensing solvents accurately and precisely is desirable.

Workstation systems capable of producing sample preparations are known though the volumes are limited. The literature contains a report of a design for a computer controlled mobile phase generator based upon the use of metering pumps. The reported accuracy of the system was +/−2%, which is 10 times greater than a typical Class A, 1 liter graduated cylinder. A system that meets or exceeds the accuracy level of a typical Class A, 1 liter graduated cylinder would be desirable, particularly when generating mobile phase/solutions in amounts greater than 10 ml, such as 100 ml, more particularly 0.5 liters upwards to about 5 liters.

U.S. Pat. No. 6,756,069 describes a device for dispensing dual component liquids or concentrates packaged in separate containers. The liquids or concentrates can be dispensed through a pumping system that is preferably a peristaltic pump and mixed together to provide a consumable beverage. The pump systems described therein rely upon volumetric displacement calculations and corrections to produce the desired consumable beverage.

U.S. Pat. No. 6,422,465 describes a process and apparatus for the preparation of custom blended fuels. A bar code is scanned by a bar code reader to convey information about a fuel required or desired to a computer controlled customized blender associated with the fuel dispensing means. The metering means are volumetrically based pumps, such as positive displacement pumps, sliding vane pumps or other positive displacement pumps that dispense a predetermined amount of volume with each stroke or rotation.

A system would be desirable that is capable of generating solutions having a high degree of accuracy but requiring little in the way of user intervention. Additionally, a system would be desirable that is capable of producing clinical formulations and buffer solutions by dissolving solids without having to measure the mass of solid ingredients before placing in collection vessel.

### SUMMARY OF THE INVENTION

For the purposes of the present invention the generic term high throughput high performance chromatography (HT-HPC) is defined to include high throughput high performance liquid, gas, capillary, microbore, preparative, electro, and supercritical fluid chromatography systems.

The present invention is directed to an automatic solution generator suitable for, as an example, a mobile phase for use with HT-HPC systems and methods that solves the aforementioned problems and disadvantages associated with conventional solution generator systems and techniques.

The present invention relates to an automated solution generator having at least one solution reservoir, a solution vessel in fluid communication with the at least one solution reservoir, a mass balance on which the solution vessel rests; and a process control system that is in communication with the mass balance to periodically receive readings of the mass of the solution vessel, the contents of the solution vessel, or the combined mass of the solution vessel and its contents. The process control system regulates the flow into the solution vessel based upon the periodic readings communicated by the mass balance. A valve can be provided between the at least one solution reservoir and the solution vessel that is in communication with the process control system and regulates the flow of solution. The automated solution generator can further comprise a data repository for the properties of a plurality of solutions and/or the name of the solution supplier.

The process control system preferably uses an iterative mass flow calculation to determine amount of solution added to the solution collection vessel. The solution collection vessel can capable of holding a solution volume of at least 10 milliliters. Alternatively, the solution collection vessel can be capable of holding a solution volume of between 0.5 and 5 liters.

In a further aspect, the automated solution generator system can further having a printer in communication with the process control system. The printer can be requested to produce documentation and labeling suitable for process control, inventory management and quality control auditing.

In a still further aspect, the automated solution generator system has a plurality of solution reservoirs to allow for the preparation of various solution compositions and mixtures of such solutions. Each of solution reservoirs is preferably individually in fluid communication with the solution collection reservoir to avoid cross-contamination issues.

The present invention also relates to methods for making a solution by introducing at least one solution into a solution collection vessel and regulating the total amount of solution introduced into the solution collection vessel using a process control system based on a series of mass-based calculations. The process control system can utilize an iterative mass calculation to estimate the amount of solution added to the solution vessel.

In a further aspect of the invention, a printer is in communication with the process control system. The printer can produce labels showing, for example, the actual amount of solution dispensed or a similar measure relative to a previously defined amount of solution to be added.

In a further aspect of the invention, the method relates to the production of solutions having a volume of at least 10 milliliters. The amount of solution added to the solution collection vessel preferably does not exceed five (5) liters.

The present invention further relates to methods of making solutions that include the step of introducing a solid ingredient into the solution collection vessel before introducing a liquid thereto. A desired solution concentration for the solid ingredient can be imported into a process control system and thereby allow the process control system to calculate a required amount of solution to be introduced into the solution vessel.

### BRIEF DESCRIPTION OF THE DRAWINGS

The foregoing and other features of the present invention will be more readily apparent from the following detailed description and drawings of illustrative embodiments of the invention wherein like reference numbers refer to similar elements throughout the several views and in which:

FIG. 1 is a schematic diagram of an automated mobile phase generator system.

FIG. 2 is a diagram of a bottle top useful for the practice of the present invention.

FIG. 3 is a flow diagram of a process control system useful for the practice of the present invention.

### DETAILED DESCRIPTION OF THE INVENTION

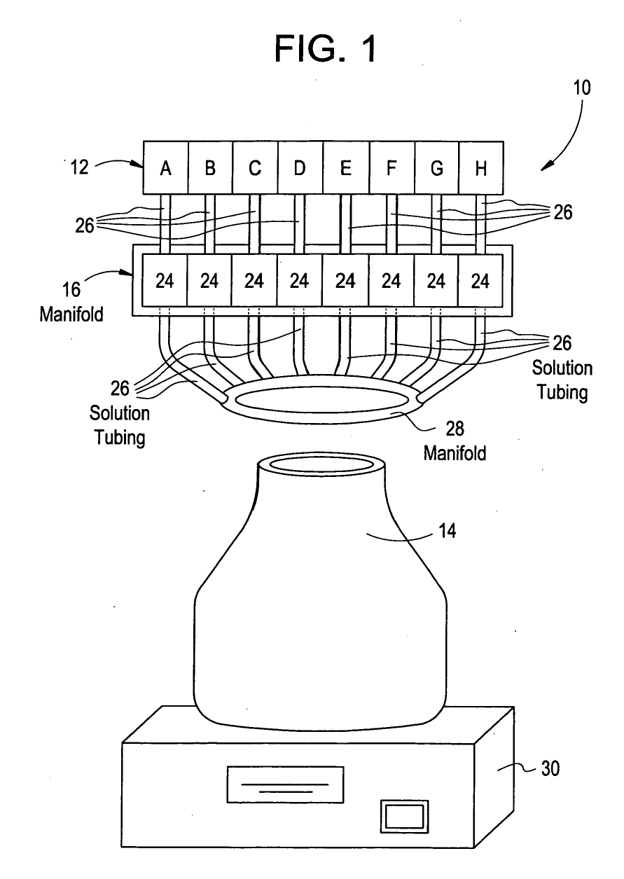

FIG. 1 is an exemplary automated solution generator (ASG) system 10 in accordance with the present invention. ASG system 10 includes a plurality, exemplified herein as 8, pressurized solution reservoirs 12 that are available to provide one or more solutions A-H, respectively, to a solution collection vessel 14 through a multi-valve manifold 16. Although the present invention is shown and described with respect to an ASG system 10 having eight (8) solution reservoirs and corresponding solutions and a corresponding number of valves in manifold 16, it is within the intended scope of the present invention to use any number of desired reservoirs, solutions and valves. Further, those skilled in the art will understand that a desired solution can consist of one or a mixture of solutions.

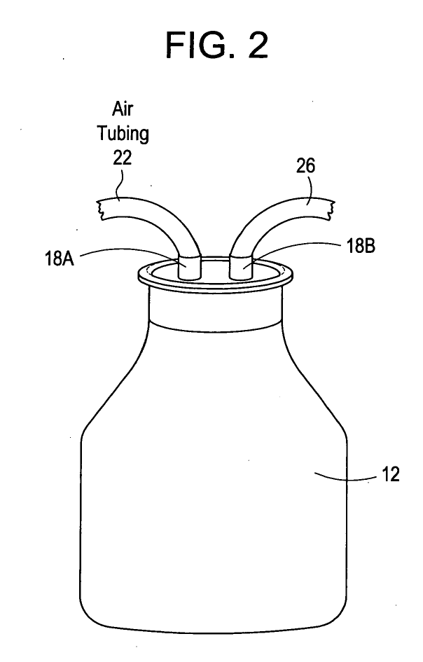

Each solution reservoir is fitted with a two-port (18A and 18B) bottle cap 20 (commercially available from Biafore Cad Concepts, Pipersville, Pa.) via a nut and ferrule. See FIG. 2. First, Port 18A is connected to a multi-valve air manifold (not shown), such as Model 5469K173, available from McMaster-Carr, New Brunswick, N.J.) via air tubing 22. A positive pressure is applied to the headspace of each solution reservoir 12 with nitrogen gas (or other inert gas) from an in-house gas source. Higher pressure levels have been found to increase flow rate of solution through ASG system 10 holding all other parameters constant. However, increasing pressure was also found to reduce dispensing accuracy. Applicants found that a positive pressure of about 14 psig produces the best combination of speed of delivery and accuracy. A gas regulator, such as Model R352-02ASS, Watts Fluidic Inc., Kittery, Me.) can be used to control the gas pressure. Further, to prevent over pressurization of solution reservoirs 12, one port of the multi-valve air manifold can be fitted with a pressure release valve set at a desired pressure setting, such as 20 psig. Pressure release valve are known and commercially available.

Second port 18B for each solution reservoir 12 is connected to a corresponding control valve 24(A-H), preferably a two-way solenoid valve, via solution tubing 26. Each control valve 24 has an inlet and an outlet port. Both solution tubing 26 and air tubing 22 are preferably constructed using a high purity Teflon PFA (perfluoro alkoxy alkane) material. Such tubing is known to be compatible with conventional HPLC solvents and is commercially available.

The outlet port from control valve 24 is connected to a solution delivery tube 26 that is connected to a solution delivery manifold 28. Solution delivery manifold 28 is suspended from multi-valve manifold 16 and is centered above an analytical balance 30. Solution delivery manifold 28 can preferably be height adjustable to accommodate solution collection vessels 14 of various volumes and dimensions.

Solution delivery tubes 26 (A-H) are affixed to solution delivery manifold 28 in such a way to preferably avoid cross-contamination of solutions. Additionally solution delivery tubes 26 (A-H) can be affixed at an angle such that the delivered solution strikes the inside wall of solution collection vessel 14. Analytical balance 30 provides in conventional fashion a measure of the mass of solution collection vessel 14, the contents of solution collection vessel 14 or the combined mass of the solution collection vessel 14 and its contents. Those skilled in the art understand that analytical balance 30 can be calibrated or tare weighted at any time to determine the change in mass of the contents of solution collection vessel 14. Analytical balance 30 provides a data feedback loop during solution delivery for the computer control logic system described below. Computer control of the solution delivery process and data exchange is accomplished using RS-232 communication (FP-1000, National Instruments, Austin, Tex.), a Labview-based control logic (Process Automation, Belle Mead, N.J.), and a relay fieldpoint module (FP-RLY-420, National Instruments, Austin, Tex.). While a particular process control system has been described above, many other process control and data feedback systems are commercially available.

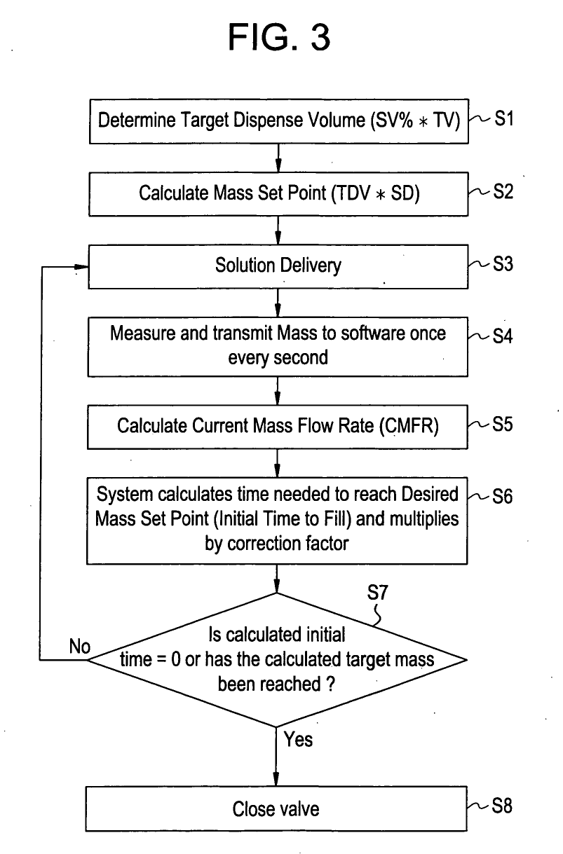

The process control system preferably shown a flowchart in FIG. 3 utilizes a software program using Labview 7.0 (National Instruments, Austin, Tex.) and is preferably accessible by a user as a graphic interface. Many similar software programs and languages are available for designing a process control system suitable for practicing this invention. The process control system should at a minimum have the ability to capture desired solution characteristics and store this information in a database. Further, the process control system should have access to physical characteristics of conventional solvents intended for use in preparing the solutions. The system should be capable of analyzing user entered criteria and database information to generate filling sequences to collect real-time process conditions to control system components. Finally, the process control system can be fully integrated into a network or used on a stand-alone basis for monitoring inventory levels, labeling containers, and reporting solvent consumption for regulatory and laboratory management purposes.

To begin, a desired solution is first defined from a list of solvents or solutions available from a database accessible by the process control system. The ASG system 10 shown in FIG. 1 can accommodate up to 8 different solutions as components for the preparation of a single mobile phase. The process control system can be designed to work with more or less number of solutions depending on the needs of a particular facility. After selecting the desired mobile phase constituents, the volume percentage, preferably in a range of 5 to 100%, of each component is defined and entered into a program database along with the total volume of mobile phase to be prepared, preferably in the range of 500 mL to 4 L. It is readily apparent that the volumetric amounts for the individual components could be entered, rather than percent by volume. In addition to the above stated information, the process control system can require that a descriptive mixture name and the name of the individual operating the workstation be entered into a predefined field and recorded in a database before the automated preparation can begin.

The process control system can have access to a database containing the densities of commonly used solution components. The database should preferably be accessible by selected individuals to allow for additional stock solvents and corresponding densities, as needed, or other information needed for process control, inventory management, regulatory reporting or internal tracking purposes. Alternatively, the user can enter the physical properties of the desired solutions manually into the process control system.

Once the required information is entered into the database, the process control system is ready to begin preparing a requested solution. The control software can be designed to dispense one or more solution components at a time. For control purposes, it has been found that dispensing a single solution component is preferred. The process control software program generates a filling sequence. The order of filling is typically not material and can preferably be determined by the user. From this point forward, all dispensing functions and calculations are performed according to the filling sequence.

A selected solution collection vessel 14 is placed on the center of analytical balance 30. The dispensing routine begins with the control software signaling analytical balance 30 to tare. A target dispense volume of a first selected solution is determined by multiplying the requested volume percentage by the total volume of solution desired. The mass set point is, for purposes of this application, defined as the mass equivalent to the volume of each component to be added to the solution. The mass set point can be calculated using the total volume of the solution, the desired volume percentage for the selected component, and its density as entered in the process control system database. A mass set point for the first selected component is calculated by multiplying the calculated target dispenses volume by the solution's density. As noted above, a user could enter the actual volume rather than a volumetric percentage. Consequently, the first step of the foregoing calculation could be eliminated.

The time that appropriate control valve 24 is to be opened is calculated to be shorter than the time needed to completely deliver the component needed. This programming step is preferably incorporated into the process control system to reduce the risk of over shooting the mass set point.

Once the mass set point is known, solution delivery is initiated by activating the appropriate control valve 24 resulting in fluid flow of the first selected component from solution reservoir (12A, for example) into solution collection vessel 14. Analytical balance 30 measures and transmits the mass to the process control software system. The mass weight of the contents of solution collection vessel 14 is recorded about once every second and used to calculate a current mass flow rate. Process control system calculates, based on the current mass flow rate, the time needed to reach the desired mass set point and multiplies this value by a correction factor. The correction factor is calculated by the process control system in known fashion to estimate the expected mass level at the next calculation step. This value is considered the initial time to fill. This routine is repeated incrementally in steps that are mandated by the time it takes for a stable mass reading to return from the scale, generally 2-3 seconds. At each step, the initial time to fill value is recalculated from the last known mass flow rate and again multiplied by the correction factor.

Process control system can shut the applicable control valve 24 short of the total fill time at predetermined period of time. This period of time is called the notch error and can range from 0.5 seconds to 5 seconds, preferably about 3 seconds. Process control system calculates a mass notch error value by multiplying the current mass flow rate and the selected notch error. The mass notch error is used to calculate the target mass, which is found by subtracting the mass set point by the mass notch error.

Process control system will close control valve 24 when calculated initial time to fill is zero and/or the calculated target mass is reached.

Upon reaching either the target mass, calculating a zero time to fill or both, process control system will slowly approach the desired mass set point by calculating a fractional time to fill. A fractional time to fill is calculated based on a selected percentage of the total remaining time that would be required to fill the solution collection vessel to the desired mass set point. The first fractional time to fill would be expected to be approximately the selected notch error.

Process control system will preferably use only a small fraction of the total time needed to reach the mass set point. The percent correction factor is optionally defined by the user, system administrator or via system default. The percent correction factor is the fraction of the total on time for the valve to be open, therefore adding only a fraction of the total mass required. For example, if the second time to fill required to reach the mass set point is three (3) seconds and the percent correction is 80%, the process control system will open the valve for 80% of three (3) seconds or 2.4 seconds. The process control system will then recalculate the time to fill and add 80% of that recalculated time to fill. This process will continue until the mass set-point is reached.

The complete process beginning with taring of the balance is then repeated for all other selected components of the mobile phase. A typical 2 liter mobile phase takes about 10 minutes to prepare.

Another aspect of the present invention is the documentation and laboratory management support functionality. Following solution preparation, ASG system 10 produces a label that preferably lists some or all of the following information, which is an exemplary non-limiting list: a) solution composition; b) solution component manufacturer(s), lot number(s) and CAS identifier; c) prepared solution expiration; d) component names; e) component density; f) actual delivered component volumes; g) actual delivered component masses; h) date of preparation; and i) calculated error of actual solution in volumetric or mass terms. One benefit of the documentation functionality is it allows the individual to confirm that the actually dispensed volumes are within acceptable ranges before use in a system taken for further analysis. This documentation functionality significantly improves quality control procedures and compliance auditing.

A still further aspect of the present invention is the ability to produce highly precise clinical formulations with speed and flexibility. As noted above, the process control system is guided by a mass balance calculation. Consequently, an individual can request preparation of a solution using a solid or powder. The individual can provide the solid or powder ingredient in a collection vessel without a prior mass balance. The advantage of the present invention is that the process control system can calculate the required amount of solution(s) to be added upon the receipt of user imported solution characteristics.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

### EXAMPLES

### Example 1

Generation of a HPLC Mobile Phase

The automated mobile phase preparation workstation prepares five (5) 1 liter mobile phases consisting of 60:40 acetonitrile: water. Record the mass of acetonitrile and water dispensed by the workstation for each preparation. Simultaneously, prepare five (5) 1 liter mobile phases (60:40 acetonitrile: water). Analyze each mobile phase by HPLC (Agilent 1100, Agilent Technologies, Palo Alto, Calif.) using a column test mixture (methyl, propyl, and butyl paraben in 60:40 acetonitrile:water), a 150×4.6 mm, 5-micron, Phenomenex Luna C18 column (00F-4041-E0, Phenomenex, Torrance, Calif.), and absorbance detection at 254 nm.

Using an optimized instrumental configuration (i.e. 0.062″ internal diameter dispensing tube, positive solution bank pressure of 14 psig, and a South Bend two-way normally closed solenoid valve), dispense 10 mL of water for a total of 24 trials. For the 24 trials, the average volume delivered was 9.97 mL±0.025 (at 95% confidence), ±0.033 (at 99% confidence), which corresponds to a dispensing accuracy of 0.55% by volume at the 95% interval and 0.63% by volume at the 99% interval which exceeds the specifications (tolerance=±0.1 mL or 1% by volume) set by the ASTM for a 10 mL Class A graduated cylinder.

At the above stated level of accuracy and under the worse case scenario (dispense volume=9.91 mL), the preparation of a 1 L mobile phase containing 1% Component A would result in a true concentration of 0.991% Component A which is acceptable for graduate cylinders. It should be noted that the label/preparation report always reports the true concentration of each component of the mobile phase and that the end user makes the final decision whether to accept or reject a mobile phase.

Determine dispensing accuracy and precision for solvents with lower viscosities and surface tensions than that of water (viscosity=1.00 cP at 20° C.; surface tension=72.8 dyne/cm at 20° C.) by instructing the system to dispense one (1) liter of solvent five (5) times for methanol and acetonitrile, separately.

For acetonitrile, a five (5) trial test dispensed a mass average of 790.02±0.06 grams or a volumetric average of 1000.15 mL±0.085 (at 95% confidence), ±0.112 (at 99% confidence), resulting in an average mass dispense error rate of only 0.02% if assume acetonitrile density of 0.7899 g/mL and a volumetric error rate of 0.023% at the 95% interval and 0.026% at the 99% interval.

For methanol, a five (5) trial test dispensed a mass average of 791.65 grams or a volumetric average of 1000.2 mL±0.16 (at 95% confidence), ±0.21 (at 99% confidence), resulting in an average dispense error of 0.03% if assume methanol density of 0.7866 g/mL and a volumetric error rate of 0.036% at the 95% interval and 0.041% at the 99% interval.

### Example 2

Four (4) randomly selected laboratory scientists tare a clean and empty graduated cylinder on an analytical balance. Then using the tared graduated cylinder, each measures one (1) liter of water. Record mass of water contained in the graduated cylinder and compare to the target mass of 997.1 g for one (1) liter of water. Each scientist repeats the procedure five (5) times. The five (5) trial average for the workstation's delivery was 1000.65 mL±0.41 (at 95% confidence), ±0.54 (at 99% confidence), resulting in an average percent error of 0.07% if assume the density of water of 0.9971 g/mL at 25° C.

What is claimed is:

1. An automated solution generator comprising:
(a) at least one solution reservoir;
(b) a solution vessel in fluid communication with at least one solution reservoir;
(c) a mass balance on which the solution vessel rests; and
(d) a process control system that is in communication with the mass balance to periodically receive readings of the mass of the solution vessel, the contents of the solution vessel, or the combined mass of the solution vessel and its contents,
wherein the process control system regulates the flow into the solution vessel based upon the periodic readings communicated by the mass balance.

2. An automated solution generator according to claim 1, wherein a valve is provided between at least one solution reservoir and the solution vessel that is in communication with the process control system.

3. An automated solution generator according to claim 1 further comprising a data repository for the properties of a plurality of solutions.

4. An automated solution generator according to claim 3, wherein the data repository includes the name of the solution supplier.

5. An automated solution generator according to claim 1 wherein the process control system uses an iterative mass flow calculation to determine amount of solution added to the solution collection vessel.

6. An automated solution generator according to claim 1 wherein the solution collection vessel has a volume of at least 10 milliliters.

7. An automated solution generator according to claim 5 wherein the solution collection vessel has a volume of between 0.5 and 5 liters.

8. An automated solution generator according to claim 1 further comprising a printer in communication with the process control system.

9. An automated solution generator according to claim 1 having a plurality of solution reservoirs.

10. An automated solution generator according to claim 9 wherein the plurality of solution reservoirs are each individually in fluid communication with the solution collection reservoir.

11. A method for making a solution comprising:
(a) introducing at least one solution into a solution collection vessel;
(b) regulating the total amount of solution introduced into the solution collection vessel using a process control system based on a series of mass-based calculations.

12. A method according to claim 11 wherein the process control system utilizes an iterative mass calculation to estimate the amount of solution added to the solution vessel.

13. A method according to claim 12 wherein a printer is in communication with the process control system.

14. A method according to claim 13 wherein the printer prints labels showing the actual amount of solution dispensed or a similar measure relative to a previously defined amount of solution to be added.

15. A method according to claim 11 wherein the amount of solution added is at least 10 milliliters.

16. A method according to claim 15 wherein the amount of solution added to the solution collection vessel does not exceed five (5) liters.

17. A method according to claim 11 further comprising the step of introducing a solid ingredient into the solution collection vessel before introducing a solution thereto.

18. A method according to claim 17 further comprising the step of entering a desired solution concentration for the solid ingredient and causing the process control system to calculate a required amount of solution to be introduced into the solution vessel.

19. A method according to claim 11 wherein a plurality of solutions are introduced into the solution collection vessel individually and sequentially.
